(19) United States
(12) Patent Application Publication
(10) Pub. No.: US 2022/0273210 A1
(43) Pub. Date: Sep. 1, 2022

(54) METHOD FOR DETERMINING PREFERENCE, AND DEVICE FOR DETERMINING PREFERENCE USING SAME

(71) Applicant: LOOXID LABS INC., Daejeon (KR)

(72) Inventors: Hong Gu LEE, Seoul (KR); Song Sub LEE, Seoul (KR)

(73) Assignee: LOOXID LABS INC., Daejeon (KR)

(21) Appl. No.: 17/638,824

(22) PCT Filed: Apr. 16, 2020
(86) PCT No.: PCT/KR2020/005071

§ 371 Date: Feb. 26, 2022

(30) Foreign Application Priority Data
Aug. 29, 2019 (KR) 10-2019-0106883

(51) Int. Cl. A61B 5/16; A61B 5/00; A61B 5/369

(52) U.S. Cl. CPC A61B 5/163; A61B 5/725; A61B 5/369

(57) ABSTRACT

The present disclosure provides a method for determining preference implemented by a processor, comprising: providing image content to a user; receiving electroencephalogram (EEG) data and gaze data including a series of gaze position data or gaze speed data which is measured while the content is provided; determining the user's region of interest with respect to the content based on the gaze data; determining a saccade onset time based on the gaze data; extracting EEG data during a time period including the saccade onset time, based on the EEG data; and determining whether the user prefers the region of interest based on the EEG data during the time period, and a device for determining preference using the same.

### TECHNICAL FIELD

The present disclosure relates to a method for determining preference and a device for determining preference using the same, and more particularly, to a method for determining preference, which determines and provides whether there is a user's preference with respect to image content based on bio-signal data, and a device for determining preference using the same.

### BACKGROUND ART

Neuromarketing is a compound word of neurons which are nerves transmitting information, and marketing, and neuromarketing may mean, after analyzing emotions and purchasing behaviors from consumers' unconscious through neuroscience, applying them to marketing. This neuromarketing is being used variously to measure marketing effects by measuring consumers' psychology and emotional responses. For example, neuromarketing is being studied as a convergence study with neuroscience in various fields such as product design, architecture, sports, and advertising marketing, and through the neuromarketing, study subjects such as products, advertisements and brands that influence marketing can be measured quantitatively to thereby find out the degree of influence they have on purchase decisions of consumers.

Meanwhile, neuromarketing can measure and analyze human bio-data such as autonomic nervous system response, indicate figures thereof using various statistical techniques, and analyze human behavior affecting marketing. In this case, as measurement of the bio-data, there may be functional magnetic resonance imaging (FMRI), electroencephalogram (EEG) measurement, eye tracking, and the like.

In conventional neuromarketing, single bio-data of one of functional magnetic resonance imaging, electroencephalogram measurement, and eye tracking was applied to analyze psychology and emotional responses of consumers. In conventional neuromarketing based on such single bio-data, bio-data may appear in various ways depending on individuals, and thus reliability of analysis may be low. In particular, in the case of eye tracking, it is possible to check a consumer's attention based on a degree to which the consumer's gaze stays. However, it may be difficult to specifically analyze whether the consumer actually gazes only on a gaze level, the gaze is made in a high preference state, or the gaze is made in a low preference state. Furthermore, expensive analysis equipment and professional manpower may be required for analysis of bio-signal data in the conventional neuromarketing, which may entail inconvenience.

Accordingly, for successful neuromarketing, the development of a new system capable of more accurately and specifically analyzing a consumer's psychological state such as preference or non-preference is continuously required.

The background art of the invention has been described to facilitate understanding of the present disclosure. Thus, it should not be understood as acknowledging that matters described in the background art of the invention exist in the prior arts.

### DETAILED DESCRIPTION OF THE INVENTION

The inventors of the present disclosure have noted distinction of interests with preference and interests without preference, with respect to degrees of interest associated with consumer psychology.

More specifically, a consumer may be interested in a particular product because the consumer has a high preference, or may be interested therein to avoid consumption of the product because the consumer has a high dislike.

Accordingly, the inventors of the present disclosure could recognize the importance of distinction between an interest with preference or an interest without preference in providing an accurate neuromarketing analysis result.

Meanwhile, the inventors of the present disclosure have more focused on a head mounted device (HMD) capable of providing bio-signal data including gaze data corresponding to a user's interest and providing various contents, in distinguishing whether the interest is an interest with preference or an interest without preference.

At this time, the HMD device may be a display device formed in a structure that can be worn on a user's head and provide an image in virtual reality (VR), augmented reality (AR) and/or mixed reality (MR) to the user so that the user can have a spatial and temporal experience similar to the real one. Such an HMD device may be configured of a main body which is formed in the form of goggles so as to be worn on the user's eye region, and a wearing unit which is connected to the main body and formed in a band form to fix the main body to the user's head. Furthermore, the HMD device may be provided with a sensor that obtains bio-signal data such as a user's gaze and brain waves, and further include a content output unit that outputs content requiring preference detection in virtual reality, augmented reality, and/or mixed reality.

Accordingly, the inventors of the present disclosure could recognize that a region of interest corresponding to the user's gaze can be extracted based on the user's bio-signal data according to the content provided through the HMD device, more specifically, gaze data, and whether the region of interest is preferred can be determined.

On the other hand, the inventors of the present disclosure have noted correlation between the gaze data and the bio-signal data of electroencephalogram (EEG) data in determining preference for a region of interest. In particular, the inventors of the present disclosure have noted specific points in time at which an interest with preference or without preference may be distinguished.

More specifically, the inventors of the present disclosure have found that EEG data at a saccade onset time in which the movement of gaze rapidly changes has an important characteristic value in distinguishing an interest with preference or an interest without preference.

As a result, the inventors of the present disclosure have come to develop a new system for determining preference, which determines a saccade onset time based on gaze data obtained while a specific image content is provided and extracts EEG data during a time period including the saccade onset time.

The inventors of the present disclosure could provide a system configured to distinguish and provide a user's interests according to whether there is the user's preference for the content, and could expect that limitations of the conventional neuromarketing are able to be overcome.

In particular, the inventors of the present disclosure could expect that, by providing the system, a user's consumption emotion could be inferred more sensitively and accurately based on the user's bio-signal data for a specific content.

Accordingly, an aspect of the present disclosure is to provide a method for determining preference, which is configured to receive a user's gaze data and EEG data according to a provision of image content, determine a region of interest and a saccade onset time based on the gaze data, extract the EEG data during a time period including the saccade onset time, and based on this, determine whether the region of interest in the image content is preferred, and a device using the same.

It will be appreciated by those skilled in the art that objects of the present disclosure are not limited to those described above and other objects that are not described above will be more clearly understood from the following descriptions.

In order to solve tasks described above, a method for determining preference according to an exemplary embodiment of the present disclosure is provided. A method for determining preference using a user's bio-signal data, which is performed by a processor according to an exemplary embodiment of the present disclosure, includes providing image content to a user; receiving electroencephalogram (EEG) data and gaze data including a series of gaze position data or gaze speed data measured while the image content is provided; determining the user's region of interest with respect to the content based on the gaze data; determining a saccade onset time based on the gaze data; extracting EEG data during a time period including the saccade onset time, based on the EEG data; and determining whether the user prefers the region of interest based on the EEG data during the time period.

According to a feature of the present disclosure, the determining of the saccade onset time may further include dividing the gaze position data into a plurality of unit periods having a predetermined time interval; and determining a saccade period including the saccade onset time in which the user's gaze rapidly changes among the plurality of unit periods, based on gaze speed data in each of the plurality of unit periods. Further, the extracting of the EEG data may include extracting EEG data corresponding to the saccade period.

According to another feature of the present disclosure, the determining of the saccade period may further include classifying each of the plurality of unit periods into the saccade period or a fixation period based on the gaze speed data in each of the plurality of unit periods; and selecting the saccade period among the plurality of classified unit periods.

According to still another feature of the present disclosure, the classifying into the saccade period or the fixation period may further include assigning a weight to at least one period among the plurality of unit periods based on the gaze speed data; and classifying each of the plurality of unit periods into the saccade period or the fixation period based on the weight.

According to still another feature of the present disclosure, the assigning of the weight may include classifying the plurality of unit periods into a first group or a second group having a lower gaze speed than the first group, based on the gaze speed data; determining a reciprocal of the number of periods belonging to the first group among the plurality of unit periods as a weight for the first group and 0 as a weight for the second group; and assigning the weights determined for each of the first group and the second group, respectively. Also, the classifying of each of the plurality of unit periods into the saccade period or the fixation period based on the weights may include determining the saccade period among the plurality of unit periods which are classified into the first group based on the weight for the first group; and determining the fixation period based on the gaze speed data for the plurality of unit periods which are classified into the second group.

According to still another feature of the present disclosure, the method of the present disclosure may further include filtering the EEG data based on at least one filter among a 0.5 Hz high filter, a 60 Hz stop filter, and a 1 to 10 Hz band pass filter, which is performed after the extracting of the EEG data during the time period including the saccade onset time.

According to still another feature of the present disclosure, the extracting of the EEG data during the time period including the saccade onset time may include extracting EEG data before and after a predetermined time based on the saccade onset time.

According to still another feature of the present disclosure, the determining of whether the user prefers the region of interest may include determining that the region of interest is preferred when the EEG data before the saccade onset time is attenuated compared to the EEG data at the saccade onset time.

According to still another feature of the present disclosure, the determining of whether the user prefers the region of interest may include determining that the region of interest is preferred when the EEG data after the saccade onset time is attenuated compared to the EEG data at the saccade onset time.

According to still another feature of the present disclosure, the method of the present disclosure may further include correcting the gaze data and the EEG data, which is performed after the receiving of the gaze data and EEG data.

According to still another feature of the present disclosure, the determining of whether the user prefers the region of interest may further include determining whether the user prefers the region of interest by using a prediction model which is configured to predict the user's preference based on the EEG data at the saccade onset time.

According to still another feature of the present disclosure, the method of the present disclosure may further include differently displaying and providing the region of interest, in the image content, according to whether the region of interest is preferred.

In order to solve tasks described above, a device for determining preference according to another exemplary embodiment of the present disclosure is provided. The device of the present disclosure includes an output unit configured to provide image content to a user; a receiving unit configured to receive EEG data, and gaze data including a series of gaze position data or gaze speed data which is measured while the image content is provided; and a processor configured to communicate with the receiving unit and the output unit. In this case, the processor is configured to determine the user's region of interest with respect to the content based on the gaze data, determine a saccade onset time based on the gaze data, extract EEG data during a time period including the saccade onset time, based on the EEG data, and determine whether the user prefers the region of interest based on the EEG data during the time period.

According to a feature of the present disclosure, the processor may be further configured to divide the gaze position data into a plurality of unit periods having a predetermined time interval, and determine a saccade period including the saccade onset time in which the user's gaze rapidly changes among the plurality of unit periods, based on gaze speed data in each of the plurality of unit periods, and extract EEG data corresponding to the saccade period.

According to another feature of the present disclosure, the processor may be further configured to classify each of the plurality of unit periods into the saccade period or a fixation period based on the gaze speed data in each of the plurality of unit periods, and select the saccade period among the plurality of classified unit periods.

According to still another feature of the present disclosure, the processor may be further configured to assign a weight to at least one period among the plurality of unit periods based on the gaze speed data, and classify each of the plurality of unit periods into the saccade period or the fixation period based on the weight.

According to still another feature of the present disclosure, the processor may be further configured to classify the plurality of unit periods into a first group or a second group having a lower gaze speed than the first group, based on the gaze speed data, determine a reciprocal of the number of periods belonging to the first group among the plurality of unit periods as a weight for the first group and 0 as a weight for the second group, assign the weights determined for each of the first group and the second group, respectively, determine the saccade period among the plurality of unit periods which are classified into the first group based on the weight for the first group, and determine the fixation period based on the gaze speed data for the plurality of unit periods which are classified into the second group.

According to still another feature of the present disclosure, the device may further include a filter unit configured to filter the EEG data based on at least one filter among a 0.5 Hz high filter, a 60 Hz stop filter, and a 1 to 10 Hz band pass filter.

According to still another feature of the present disclosure, the processor may be configured to extract EEG data before and after a predetermined time based on the saccade onset time.

According to still another feature of the present disclosure, the processor may be configured to determine that the region of interest is preferred when the EEG data before the saccade onset time is attenuated compared to the EEG data at the saccade onset time.

According to still another feature of the present disclosure, the processor may be configured to determine that the region of interest is preferred when the EEG data after the saccade onset time is attenuated compared to the EEG data at the saccade onset time.

According to still another feature of the present disclosure, the processor may be further configured to correct the gaze data and the EGG data.

According to still another feature of the present disclosure, the processor may be further configured to determine whether the user prefers the region of interest by using a prediction model which is configured to predict the user's preference based on the EEG data at the saccade onset time.

According to still another feature of the present disclosure, the output unit may be further configured to differently display and provide the region of interest, in the image content, according to whether the region of interest is preferred.

Details of other embodiments are included in the detailed description and drawings.

The present disclosure provides a new system for determining preference that determines a saccade onset time based on gaze data obtained while image content is provided and extracts EEG data at the saccade onset time, thereby having effects capable of specifically classifying and providing a consumer's interests associated with consumer psychology.

More specifically, the present disclosure has an effect of distinguishing and providing a user's interest with a high preference for a specific product or the user's interest for avoiding consumption of the product because the user has a high dislike.

In particular, the present disclosure can more sensitively and accurately infer a user's consumption emotion based on bio-signal data such as the user's gaze data and EEG data with respect to a specific content. Accordingly, the present disclosure can provide accurate neuromarketing analysis results and overcome the limitations of conventional neuromarketing.

Furthermore, according to the present disclosure, as bio-signal data obtainable through an HMD device is used, expensive analysis equipment and professional manpower are not required, and it has an effect capable of determining whether or not there is a user's preference regardless of a location.

Effects according to the present disclosure are not limited by the content exemplified above, and more various effects are included in the present disclosure.

### BRIEF DESCRIPTION OF THE DRAWINGS

FIG. 1A is a schematic view illustrating a system for determining preference using bio-signal data according to an exemplary embodiment of the present disclosure.

FIG. 1B is a schematic diagram illustrating a device for determining preference according to an exemplary embodiment of the present disclosure.

FIG. 2 is a schematic flowchart for explaining a method for determining whether there is preference based on a user's bio-signal data according to a method for determining preference according to an exemplary embodiment of the present disclosure.

FIG. 3A exemplarily illustrates gaze data of a user which is generated by providing image content according to a method for determining preference according to an exemplary embodiment of the present disclosure.

FIGS. 3B and 3C exemplarily illustrate a step of determining a saccade onset time at which a user's gaze rapidly changes according to a method for determining preference according to an exemplary embodiment of the present disclosure.

FIG. 3D exemplarily illustrates a step of determining whether there is a user's preference according to a method for determining preference according to an exemplary embodiment of the present disclosure.

FIGS. 4A to 4E are exemplarily illustrate a user's regions of interest according to a provision of image content through a HMD device and whether there is the user's preference, which is determined with respect to the regions of interest, according to a method for determining preference according to an exemplary embodiment of the present disclosure.

Advantages and features of the present disclosure and methods to achieve them will become apparent from descriptions of exemplary embodiments herein below with reference to the accompanying drawings. However, the present disclosure is not limited to the exemplary embodiments disclosed herein but may be implemented in various different forms. The exemplary embodiments are provided to make the description of the present disclosure thorough and to fully convey the scope of the present disclosure to those skilled in the art. It is to be noted that the scope of the present disclosure is defined only by the claims.

Although terms such as first, second, and the like are used to describe various components, these components are not limited by these terms, of course. These terms are only used to distinguish one component from another component. Therefore, it goes without saying that a first component mentioned below may be a second component within the spirit of the present disclosure.

The same or like reference numerals refer to the same or like elements throughout the specification.

Features of various exemplary embodiments of the present disclosure may be partially or fully combined or coupled, and as will be clearly appreciated by those skilled in the art, technically various interactions and operations are possible, and respective exemplary embodiments may be implemented independently of each other or may be implemented together in an associated relationship.

In the present disclosure, a system for determining preference is not limited to, and may include any device which is configured to obtain a user's gaze and obtain bio-signal data such as electroencephalogram (EEG) of the user. For example, the system for determining preference may include, a device including a sensor which is in contact with/is worn on a part of the user's body and obtains the user's bio-signal data, such as a headset, a smart ring, a smart watch, an earset, an earphone, or the like; a content output device for outputting image content for which preference detection is required in association with virtual reality, augmented reality, and/or mixed reality, and an electronic device for managing them, as well as an HMD device. For example, if the HMD device has an output unit, the system for determining preference may include the HMD device and an electronic device. Here, the bio-signal data represents various signals generated from a user's body according to the user's conscious and/or unconscious (e.g., respiration, heartbeat, metabolism, etc.) actions such as the user's gaze, EGG, pulse, blood pressure, and the like.

Hereinafter, various exemplary embodiments of the present disclosure will be described in detail with reference to the accompanying drawings.

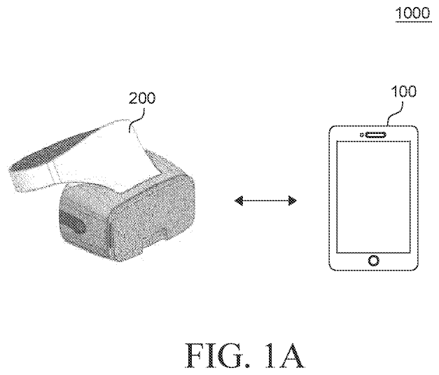

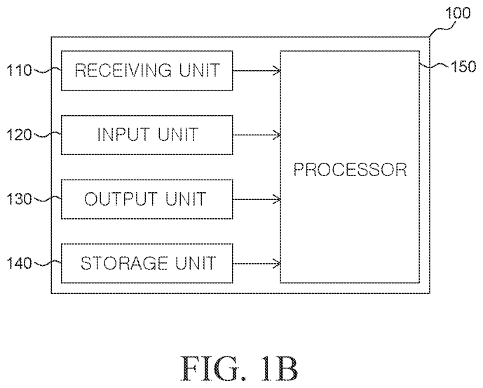

FIG. 1A is a schematic view illustrating a system for determining preference using bio-signal data according to an exemplary embodiment of the present disclosure. FIG. 1B is a schematic diagram illustrating a device for determining preference according to an exemplary embodiment of the present disclosure.

First, referring to FIG. 1A, a system 1000 for determining preference may be a system which is configured to extract a user's regions of interest based on bio-signal data including at least one of the user's electroencephalogram (EEG) data and gaze data according to a provision of image content for which preference detection is required, and classify preference for the regions of interest. In this case, the system 1000 for determining preference may be configured of a device 100 for determining preference which determines whether the user prefers based on the bio-signal data, and a head mounted display (HMD) device 200 for obtaining the bio-signal data.

In this case, the device 100 for determining preference may be communicatively connected to the HMD device 200 and may be configured to provide image content for which preference detection is required to the HMD device 200. Furthermore, the device 100 for determining preference is a device for determining preference for image content which requires preference detection and the bio-signal data obtained through the HMD device 200, and may include a personal computer (PC), a notebook computer, a workstation, a smart TV, and the like.

More specifically, referring to FIG. 1B together, the device 100 for determining preference may include a receiving unit 110, an input unit 120, an output unit 130, a storage unit 140, and a processor 150.

In this case, the receiving unit 110 may be configured to receive the user's bio-signal data according to the provision of image content for which preference detection is required. In various exemplary embodiments, the receiving unit 110 may be further configured to receive gaze data for the image content for which preference detection is required, and furthermore, EEG data during a time period in which the image content is provided.

The input unit 120 may receive settings of the device 100 for determining preference from the user. The input unit 120 may receive the user's gaze according to the provision of image content for which preference detection is required. Meanwhile, the input unit 120 may be an input unit of a head mounted display (HMD), but is not limited thereto.

The output unit 130 may be configured to provide an interface screen for confirming the user's interest and preference with respect to the image content for which preference detection is required. Here, the interface screen may include a display space for displaying the image content for which preference detection is required. Also, the output unit 130 may be configured to display and provide a region of interest in the image content determined by the processor 150, which will be described later, and whether the region of interest is preferred.

Meanwhile, the provision of the image content for which preference detection is required is not limited to the above, and may also be provided through the output unit of the HMD device 200, which will be described later.

The storage unit 140 may be configured to store various bio-signal data which is received by the receiving unit 110, the user's settings which are input through the input unit 120, and the image content for which preference detection is required, which is provided through the output unit 130. Furthermore, the storage unit 140 may be further configured to store the region of interest in the image content determined by the processor 150, which will be described later, and whether or not the region of interest is preferred. However, the present disclosure is not limited thereto, and the storage unit 140 may be configured to store all data which is generated in a process in which the processor 150 determines a degree of interest and preference with respect to the image content.

The processor 150 may be configured to determine the user's region of interest in the image content and a saccade onset time based on the gaze data and EEG data which are obtained through the HMD device 200, extract EEG data at the saccade onset time, and determine whether the determined region of interest is preferred.

In this case, the saccade onset time is a point in time at which the user's gaze rapidly changes, and may be a single point in time or may be a series of points in time in which a gaze speed of a predetermined level or more appears.

Meanwhile, the processor 150 may be configured to divide gaze position data into a plurality of unit periods having a predetermined time interval, determine a saccade period including the saccade onset time in which the user's gaze rapidly changes among the plurality of unit periods, based on gaze speed data in each of the plurality of unit periods, and extract EEG data corresponding to the saccade period.

In addition, the processor 150 may be further configured to classify each of the plurality of unit periods into a saccade period or a fixation period based on the gaze speed data in each of the plurality of unit periods, and select the saccade period among the classified plurality of unit periods.

In various exemplary embodiments, the processor 150 may be further configured to assign a weight to at least one period among the plurality of unit periods based on the gaze speed data, and based on the weight, classify each of the plurality of unit periods into the saccade period or the fixation period.

Meanwhile, the processor 150 may be configured to extract EEG data before and after a predetermined time based on the saccade onset time. In this case, the processor 150 may be configured to determine that the region of interest is preferred when the EEG data before the saccade onset time is attenuated compared to the EEG data at the saccade onset time. Furthermore, the processor 150 may be configured to determine that the region of interest is not preferred when the EEG data after the saccade onset time is attenuated compared to the EEG data at the saccade onset time.

In another exemplary embodiment of the present disclosure, the processor 150 may be further configured to correct the gaze data, and the EEG data.

Meanwhile, according to another exemplary embodiment of the present disclosure, the processor 150 may be further configured to determine whether the user prefers the region of interest by using a prediction model which is configured to predict the user's preference based on the EEG data at the saccade onset time. For example, the processor 150 may determine whether the user prefers the region of interest from various bio-data such as EGG data and gaze data, based on a deep learning algorithm. At this time, the deep learning algorithm may be at least one among a deep neural network (DNN), a convolutional neural network (CNN), a deep convolution neural network (DCNN), a recurrent neural network (RNN), a restricted Boltzmann machine (RBM), a deep belief network (DBN), and a single shot detector (SSD). Furthermore, the processor 150 may classify whether the user prefers the region of interest from various biometric data such as EGG data and gaze data, based on a classification model. In this case, the classification model may be at least one of a random forest, Gaussian Naive Bayes (GNB), a locally weighted Naive Bay (LNB), and a support vector machine (SVN). However, it is not limited to those described above, and the processor 150 may be based on more various algorithms as long as it can determine the preference based on the EEG data at the saccade onset time.

According to another exemplary embodiment of the present disclosure, the device 100 for determining preference may further include a filter unit (not shown) which is configured to filter the EGG data based on at least one filter among a 0.5 hz high filter, a 60 hz stop filter, and a 1 to 10 hz band pass filter.

Referring back to FIG. 1A, the HMD device 200 may be a complex virtual experience device which is mounted on a user's head and provides image content for virtual reality to the user so that the user can have a spatial and temporal experience similar to a real experience and at the same time, which is capable of detecting physical, cognitive, and emotional changes of the user who is undergoing a virtual experience by acquiring the user's bio-signal data. In this case, the image content which is provided through the HMD device 200 may include non-interactive images such as movies, animations, advertisements, or promotional videos, and interactive images which is mutually active with a user, such as games, electronic manuals, electronic encyclopedias or promotional videos, but is not limited thereto. Here, the image may be a 3D image, and may include a stereoscopic image.

The HMD device 200 may be formed in a structure capable of being worn on the user's head, and may be implemented in such a manner that image content requiring various preference detection is processed through the output unit inside the HMD device 200.

When the HMD device 200 includes an output unit, one surface of the output unit may be disposed to face the user's face so that the user can check the image content when the user wears the HMD device 200.

At least one sensor (not shown) which obtains the user's EGG data and gaze data may be formed on one side of the HMD device 200. The at least one sensor may include an EEG sensor that measures the user's EEG and/or an eye tracking sensor that tracks the user's gaze or stare. In various exemplary embodiments, at least one sensor is formed at a position at which the user's eyes or face can be captured or a position capable of contacting the user's skin, captures the user's eyes or face when the user wears the HMD device 200, and obtains the user's gaze data by analyzing the captured image, or obtains EEG data such as the user's electroencephalography (EEG), electromyography (EMG), or electrocardiogram (ECG) by contact with the user's skin. In this specification, the HMD device 200 is described as including at least one sensor which obtains the user's EGG data and gaze data, but is not limited thereto, and it may be implemented in a form in which at least one sensor that obtains the user's EGG or gaze data through a module separate from the HMD device 200 is mounted on an HMD housing. The expression called HMD device 200 is intended to include such a module or also contemplate the module itself.

The HMD device 200 may obtain the user's bio-signal data according to a request of the device 100 for determining preference, and transmit the obtained bio-signal data to the device 100 for determining preference through the output unit or the receiving unit.

By the system 1000 for determining preference as described above, not only the user's interest in the image content but also whether the region of interest is preferred may be determined. These analysis results can be utilized for various neuromarketing.

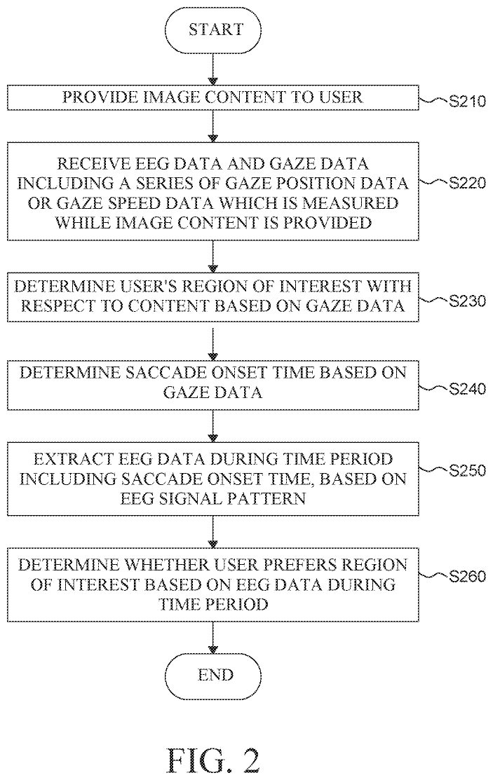

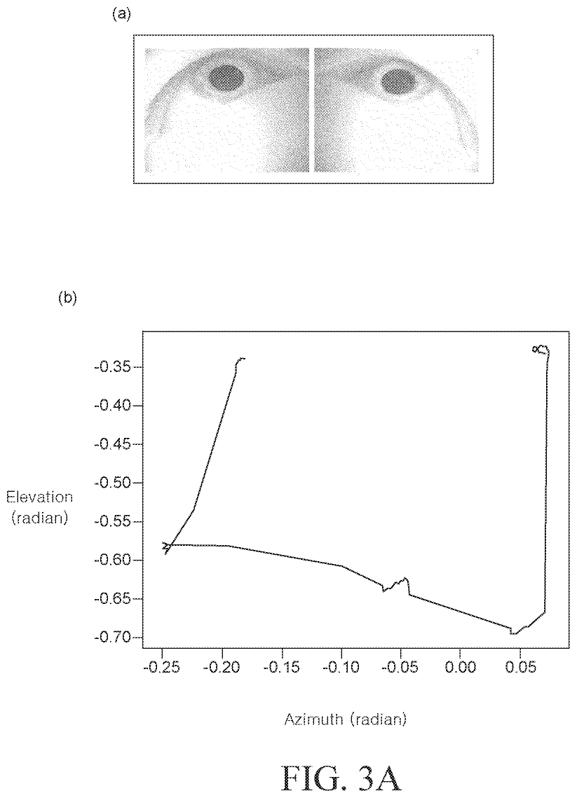

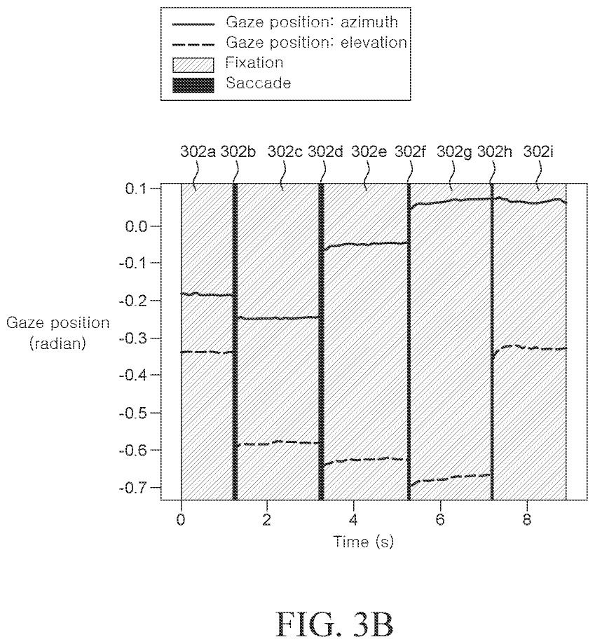

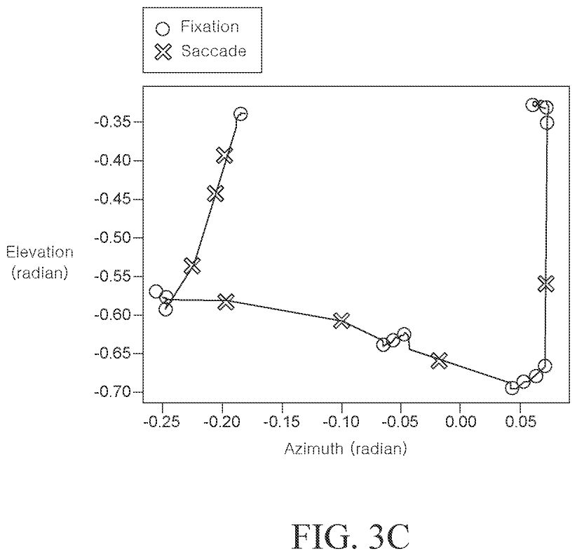

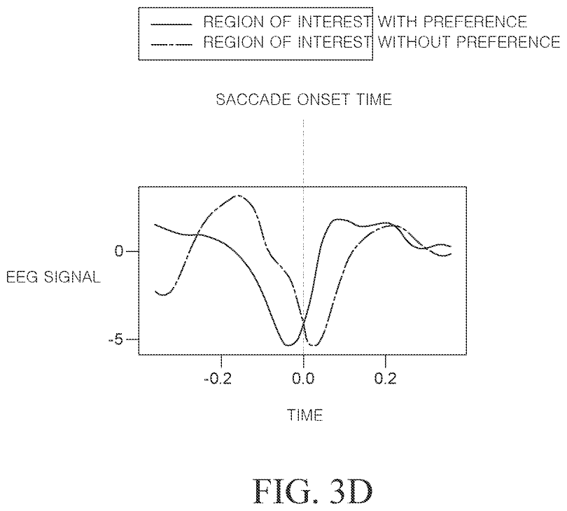

Hereinafter, procedures of a method for determining preference according to various exemplary embodiments of the present disclosure will be described with reference to FIG. 2 and FIGS. 3A to 3D.

FIG. 2 is a schematic flowchart for explaining a method for determining whether there is preference based on a user's bio-signal data according to a method for determining preference according to an exemplary embodiment of the present disclosure. FIG. 3A exemplarily illustrates gaze data of a user which is generated by providing image content according to a method for determining preference according to an exemplary embodiment of the present disclosure. FIGS. 3B and 3C exemplarily illustrate a step of determining a saccade onset time at which a user's gaze rapidly changes according to a method for determining preference according to an exemplary embodiment of the present disclosure. FIG. 3D exemplarily illustrates a step of determining whether there is a user's preference according to a method for determining preference according to an exemplary embodiment of the present disclosure.

First, according to the method for determining preference according to an exemplary embodiment of the present disclosure, image content is provided to a user in step S210. Next, EEG data and gaze data including a series of gaze position data or gaze speed data which is measured while the image content is provided, are received in step S220. Next, the user's region of interest with respect to the content is determined based on the gaze data in step S230, and the saccade onset time is determined in step S240. Next, EEG data during a time period including the saccade onset time is extracted in step S250, and finally, whether the user prefers the region of interest is determined in step S260.

More specifically, in the step S210 in which the image content is provided, the image content for which preference detection that induces an emotion of likes and dislikes to the user, is required may be provided.

According to an exemplary embodiment of the present disclosure, in the step S210 in which the image content is provided, at least one content among an image, a movie, an animation, an advertisement, a promotional video, a game, an electronic manual, an electronic encyclopedia, and a text may be provided.

Next, in the step S220 in which the gaze data and the EEG data are received, a series of data measured while the image content is provided, that is, time-series gaze data and EEG data which are obtained during a certain time period may be obtained.

At this time, according to a feature of the present disclosure, in the step S220 in which the gaze data and the EEG data are received, the gaze data including gaze at the image content for which preference detection is required may include gaze position data and gaze speed data. Furthermore, the gaze data may further include a gazing time in which gazing is made, a gaze tracking time in which the gaze tracks a specific object of the content, the number of times the user's eyes blink, and the like.

For example, referring to (a) and (b) of FIG. 3A together, in the step S220 in which the gaze data and the EEG data are received, the user's gaze position data according to the provision of the image content through the HMD device and the user's gazing may be received.

In this case, the gaze position data may be obtained based on an elevation and an azimuth of the user's gaze that changes based on a screen on which the content is provided. In this case, the obtained position of the gaze may be expressed in a unit of a plane angle (radian), but is not limited thereto. Meanwhile, the gaze position data may be obtained as an elevation and an azimuth of the gaze according to a content provision time.

According to another feature of the present disclosure, after the step S220 in which the gaze data and the EEG data are received, a step in which the received gaze data and EEG data are corrected may be further performed.

For example, in the step of correction, an EEG signal pattern associated with an interest having a predetermined preference from pre-stored data and an EEG signal pattern not associated with the interest may be corrected to be applied to a specific individual. More specifically, in the step of correction, two contents allowing the user to have contrasting emotions are provided to the user, and EEG data at a saccade onset time during gazing at each content and EEG data during a time period including the saccade onset time, that is, a series of EEG patterns can be detected. In this case, a newly detected EEG data may be mapped with the EEG signal pattern associated with an interest having a predetermined preference and the EEG signal pattern not associated with the interest. Through this process, preference for an individual user can be predicted more accurately.

Next, in the step S230 in which the user's region of interest with respect to the content is determined, the region of interest with respect to the content may be determined based on the user's gaze data. In this case, the user's gaze at the content may correspond to the user's interest.

Next, in the step S240 in which the saccade onset time is determined, the saccade onset time at which the user's gaze rapidly changes may be determined.

According to a feature of the present disclosure, in the step S240 in which the saccade onset time is determined, the gaze position data is divided into a plurality of unit periods having a predetermined time interval, and based on the gaze speed data in each of the plurality of unit periods, a saccade period including the saccade onset time among the plurality of unit periods may be determined.

According to another feature of the present disclosure, in the step S240 in which the saccade onset time is determined, each of the plurality of unit periods may be classified into the saccade period or the fixation period based on the gaze speed data in each of the plurality of unit periods, and the saccade period may be selected among the plurality of classified unit periods.

For example, referring to FIG. 3B, the gaze position data of the elevation and azimuth according to the content provision time may be divided into a plurality of time units 302a, 302b, 302c, 302d, 302e, 302f 302g, 302i and 302h in units of seconds. Then, based on gaze speed data in each of a plurality of periods, that is, gaze movement distances and times, it may be classified into saccade periods 302b, 302d, 302f, 302h and fixation periods 302a, 302c, 302e, 302g, 302i. That is, with more reference to FIG. 3C, the gaze position data may be labeled during a period in which gaze saccade or gaze fixation is performed.

In this case, classification of the saccade period and the fixation period may be performed by assigning a weight to at least one period among the plurality of unit periods based on the gaze speed data. More specifically, for each of the gaze position data which is divided into a plurality of periods, a weight may be added as a velocity of gaze for a corresponding unit period increases.

For example, for each gaze position data (x(t), y(t)) which is divided into a plurality of unit periods, for classification of the saccade period and the fixation period, by using the velocity of the gaze V(t)=\sqrt{\dot{x}(t)²°\dot{y}(t)²}, then k-means-clustering, the plurality of unit periods are classified into a first group (V₁) and a second group (V₂) having a lower speed than the first group. In this case, the two groups may be classified by Equation 1 below.

V₁={v(tᵢ¹)}, V₂={v(tᵢ²)}, E[V₁]≥E[V₂] [Equation 1]

Then, weight ω is set to a reciprocal of the number of samples belonging to the first group V₁, that is, a reciprocal of the number of periods (ω=1/|V₁|), and the weight ω may be assigned to tᵢ¹, which is a period belonging to the first group V₁. In this case, 0 may be assigned as a weight to tᵢ², which is a period belonging to the second group V₂. Next, the gaze position data of the plurality of unit periods, classified into two groups, may be classified into a saccade period and a fixation period, and furthermore, ‘unknown’ which is not classified as the saccade period and the fixation period, using an assigned weight value W(t). More specifically, when the weight value W(t) assigned in a specific period is greater than the average of weights by more than a standard deviation, the specific period may be classified as the saccade period. In the period t′ᵢ in which the weight value W(t) is 0, a period in which v(t′)<E[v(t′)] may be classified as the fixation period. At this time, in the period of ‘unknown’, when a length of a blank period is less than or equal to a predetermined level, and average velocities of the gaze position immediately before and immediately after the blank period are v̅1, v̅2, respectively, in a case in which an average velocity v̅B of the blank period satisfies |v̅1−v̅2|>min [|v̅1−v̅B|, |v̅2−v̅B|], it can be included in a period close to the average velocity among the two groups.

Meanwhile, the step S240 in which the saccade onset time is determined is not limited to the above-described method, and may be performed in more various methods.

Next, in the step S250 in which EEG data is extracted, EEG data corresponding to the saccade period including the saccade onset time may be extracted.

In this case, the saccade period may mean a time period before (e.g., 0.3 seconds before the gaze saccade) and/or after (0.3 seconds after the gaze saccade) the saccade onset time.

According to another feature of the present disclosure, in the step S250 in which EEG data is extracted, EEG data before and after a predetermined time based on the saccade onset time may be extracted.

Finally, in the step S260 in which whether it is preferred is determined, whether the user prefers the region of interest in the content determined as a result of the step S230 in which the region of interest is determined may be determined.

According to a feature of the present disclosure, in the step S260 in which whether it is preferred is determined, the preference for the region of interest may be determined according to a characteristic of EEG data.

For example, referring to FIG. 3D, in the step S260 in which whether it is preferred is determined, when the EEG data before the saccade onset time (e.g., −0.2 seconds) is attenuated compared to the EEG data at the saccade onset time (e.g., 0.0), it may be determined that the region of interest is preferred. Furthermore, in the step S260 in which whether it is preferred is determined, when the EEG data after the saccade onset time (e.g., 0.2 seconds) is attenuated compared to the EEG data at the saccade onset time (e.g., 0.0), it may be determined that the region of interest is not preferred.

According to another feature of the present disclosure, in the step S260 in which whether it is preferred is determined, whether the user prefers the region of interest may be determined based on the EEG data at the saccade onset time, by using a prediction model configured to predict the user's preference.

At this time, the prediction model is a model configured to classify preference using the user's EGG data which is obtained at the saccade onset time as learning data, and may be configured to classify whether the region of interest is preferred or is not preferred based on EGG patterns. Meanwhile, the prediction model may be a model configured to predict (classify) preference based on a deep learning algorithm or a classification model. For example, in the step S260 in which whether it is preferred is determined, a prediction model based on at least one among a deep neural network (DNN), a convolutional neural network (CNN), a deep convolution neural network (DCNN), a recurrent neural network (RNN), a restricted Boltzmann machine (RBM), a deep belief network (DBN), and a single shot detector (SSD) may be used. Furthermore, in the step S260 in which whether it is preferred is determined, a prediction model based on at least one of a random forest, a Gaussian Naive Bayes (GNB), a locally weighted Naive Bay (LNB), and a support vector machine (SVN) may be used.

However, the present disclosure is not limited thereto, and in the step S260 in which whether it is preferred is determined, as long as the preference can be determined based on the EEG data at the saccade onset time, models based on more various algorithms may be applied.

According to a feature of the present disclosure, based on a result of the step S260 in which whether it is preferred is determined, a step of differently displaying and providing a region of interest in the image content depending on whether the region of interest is preferred may be further performed.

For example, in the step of differently displaying and providing the region of interest depending on whether the region of interest is preferred, the region at which the user gazes in the content may be output in a red color as a degree of interest is higher, and in a blue color as the degree of interest is lower.

In this case, the preference for the region of interest may be distinguished by indicating the region of interest as O (with preference) or X (without preference) depending on whether the region of interest is preferred.

Meanwhile, a method of displaying degrees of interest and preference is not limited thereto. For example, in the step of differently displaying and providing the region of interest depending on whether the region of interest is preferred, regions of interest in the content may be displayed as regions having a single color or pattern. In this case, when the determined regions of interest have a single color, whether or not the regions of interest are preferred may be distinguished by differentiating and displaying patterns. Furthermore, when the determined regions of interest have a single pattern, whether or not they are preferred may be distinguished by differentiating and displaying colors. In addition, when the regions of interest have a single color, whether or not they are preferred may be distinguished by differentiating and displaying the saturation, contrast, brightness, and the like thereof.

As described above, by the method for determining preference according to various exemplary embodiments of the present disclosure, preference for a region of interest in content which is provided to a user may be determined, and may be displayed and provided in the content.

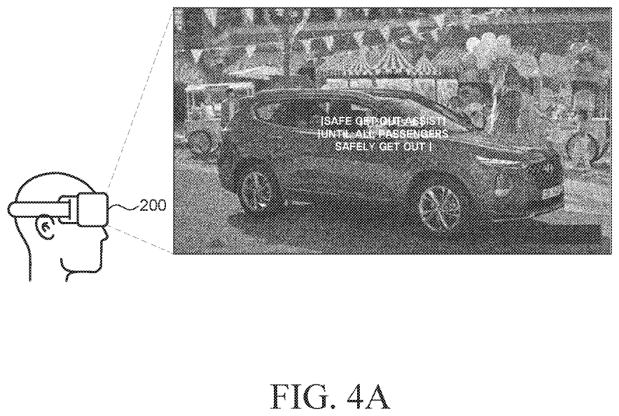

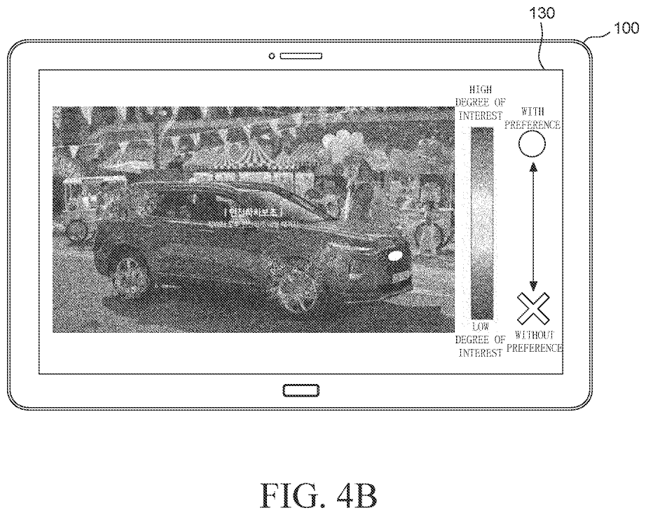

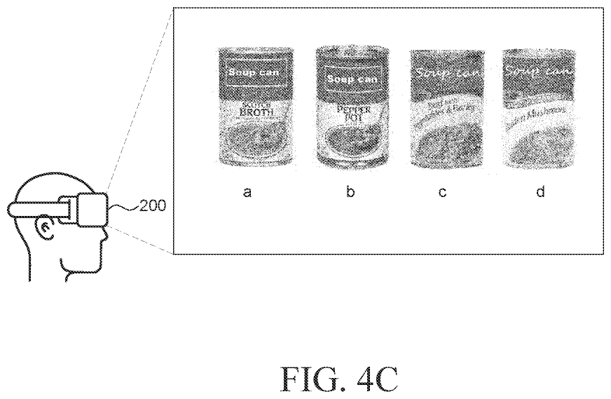

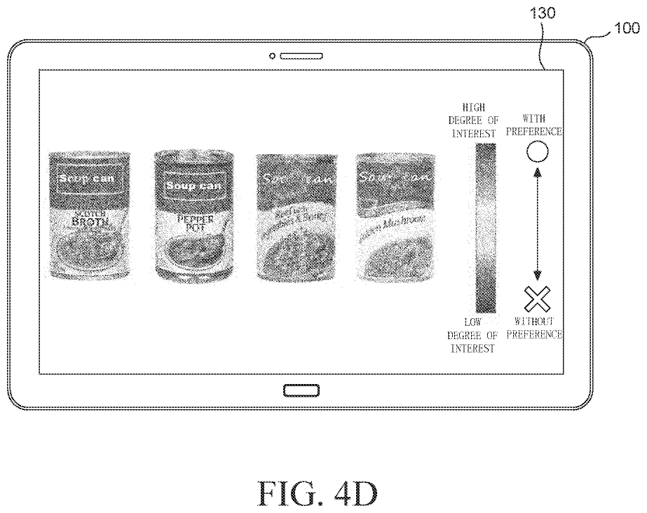

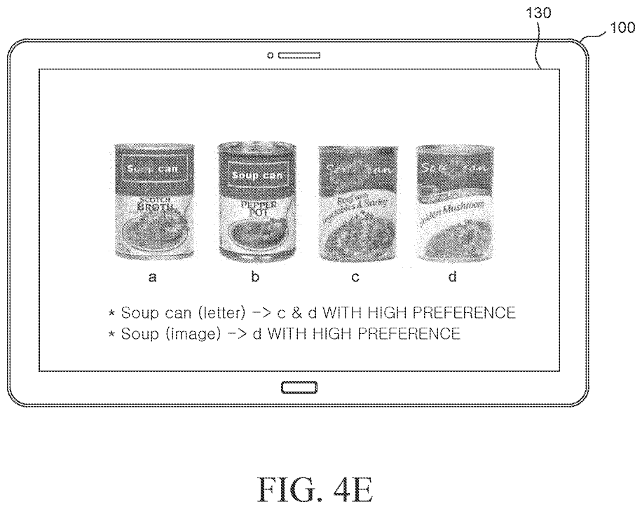

Hereinafter, procedures for determining preference for regions of interest determined by a method for determining preference according to various exemplary embodiments of the present disclosure will be exemplarily described with reference to FIGS. 4A to 4E.

FIGS. 4A to 4E are exemplarily illustrate a user's regions of interest according to a provision of image content through a HMD device and whether there is the user's preference, which is determined with respect to the regions of interest, according to a method for determining preference according to an exemplary embodiment of the present disclosure.

First, referring to FIG. 4A, the user is provided with image content for which preference detection is required through the HMD device 200. In this case, the image content may include one or more objects on which whether it is preferred is to be confirmed.

More specifically, the user may check a vehicle advertisement displayed through the output unit of the HMD device 200. The user may gaze at various objects such as a car and a background while the vehicle advertisement is provided, and by an eye tracking sensor and an EEG measurement sensor which are pre-mounted in the HMD device 200, gaze data and EEG data may be obtained while the vehicle advertisement is provided to the user. The obtained gaze data and EGG data may be received by the device 100 for determining preference of the present disclosure.

Next, referring to FIG. 4B, the output unit 130 of the device 100 for determining preference according to the present disclosure is illustrated. In this case, the output unit 130 may distinguish and display a degree of interest and whether there is preference according to the user's gaze at the image content, and provide them.

More specifically, it is shown that the user gazed at a portion of the vehicle's headlight part, front wheel, rear wheel, rear part, and background while the vehicle advertisement is presented. That is, the user's gaze region may mean a portion in which the user has a high degree of interest in the vehicle advertisement. In this case, the output unit 130 may output a red color as the degree of interest in a region gazed by the user is high, and a blue color as the degree of interest in a region gazed by the user is low. In this case, the output unit 130 may display the region of interest as O (with preference) or X (without preference) according to whether it is preferred or not. That is, according to output results, an indication of O appears on the headlight part, the front wheel, and the rear wheel of the vehicle with a high degree of interest, which indicates that the user has a high preference for the headlight part, the front wheel, and the rear wheel of the vehicle. In contrast, an indication of X appears on the rear part of the vehicle with a high degree of interest, which indicates that the user has a high degree of interest in the rear part of the vehicle but has a low preference.

Such results may mean that the user has an overall interest in the vehicle in the advertisement provided, particularly has a high preference for the headlight part and the wheel part, and has a low preference for a design such as the rear part of the vehicle.

As described above, by the device 100 for determining preference of the present disclosure, the degree of interest and whether there is preference according to the user's gaze at the image content may be analyzed and provided, and analyzed results may be further utilized for advertising marketing.

Referring to FIG. 4C, in another exemplary embodiment of the present disclosure, a user is provided with image content for which preference detection is required, through the HMD device 200. In this case, the image content may include a plurality of objects to confirm whether they are preferred. More specifically, the user may be provided with soup cans a, b, c, and d of various designs through the output unit of the HMD device 200. The user may gaze at various object regions such as product names, fonts, designs, and soup images of the soup cans while the soup cans are provided. In this case, gaze data and EGG data while the soup cans are provided to the user may be obtained by the eye tracking sensor and the EGG measurement sensor pre-mounted in the HMD device 200. Next, the gaze data and EGG data obtained by the HMD device 200 may be received by the device 100 for determining preference of the present disclosure.

Next, referring to FIG. 4D, the output unit 130 of the device 100 for determining preference of the present disclosure is shown. More specifically, it is shown that the user gazed at all soup cans a, b, c and d while the soup cans are presented. As the output unit 130 may output a red color to a certain region as a degree of interest is higher and a blue color to a certain region as a degree of interest is lower, it is shown, according to output results, that the user intensively gazed at the product names of the soup cans, the soup images shown below the product names. Furthermore, as the output unit 130 may display the region of interest as O (with preference) or X (without preference) depending on whether it is preferred, it is shown, according to the output results, that the user has a high preference for the product name of the soup can c, the product name of the soup can d, and the soup image of the soup can of d. In contrast, it is shown that the user has a high degree of interest in the soup images of the soup cans a and c but have a low preference therefor.

Such results may mean that the user has a high preference for the fonts of the product names of the soup cans a and b, and the soup image of the soup can d, compared to the other soup cans. Furthermore, it may mean that the user has a low preference for the soup images of the soup cans a and c.

That is, the device 100 for determining preference according to the present disclosure may distinguish and display a degree of interest and whether there is preference according to the user's gaze at the image content, and provide them. Meanwhile, the device 100 for determining preference may output and provide more various information.

Referring to FIG. 4E together, the device 100 for determining preference may be further configured to output and provide information on preference based on the user's regions of interest determined in the content and whether they are preferred. More specifically, as described above in connection with FIGS. 4C and 4D, in the case of the soup cans for which the regions of interest and preference were determined, the soup cans c and d with a high degree of interest and preference for the product names of the soup cans, could be output as having a high preference for letters. Furthermore, the soup can d with a high degree of interest and preference for the soup image may be output as having high preference for the soup image.

As described above, by the device 100 for determining preference of the present disclosure, the degree of interest and whether there is preference according to the user's gaze at the image content are analyzed, and various information about the preference can be provided. In this case, analyzed results may be further utilized in marketing to determine a product name, image, and the like.

On the other hand, the output of preference by the device 100 for determining preference according to the present disclosure is not limited to the indication of O or X which mentions the preference for the region of interest. For example, the device 100 for determining preference may display regions of interest in the content as regions having a single color or pattern. At this time, when determined regions of interest have a single color, the device 100 for determining preference may be configured to distinguish and output whether they are preferred by differentiating and displaying patterns, and when determined regions of interest have a single pattern, it may be configured to distinguish and output whether they are preferred by differentiating and displaying colors. In addition, when the regions of interest have a single color, whether they are preferred may be distinguished by differentiating and displaying the saturation, contrast, brightness, and the like thereof.

A method for determining preference and a device for determining preference according to an exemplary embodiment of the present disclosure may be implemented in the form of program instructions that can be executed through various computer means and recorded in a computer readable medium. The computer readable medium may include program instructions, data files, data structures, and the like, alone or in combination.

The program instructions recorded on the computer readable medium may be those specially designed and configured for the present disclosure, or may be those known and available to a person having ordinary skill in the computer software field. Examples of the computer readable recording medium include magnetic media such as hard disks, floppy disks and magnetic tapes, optical media such as CD-ROMs and DVDs, magneto-optical media such as floptical disks, and hardware devices specially configured to store and execute program instructions, such as ROMs, RAMs, flash memories, and the like. In addition, the above-mentioned medium may be a transmission medium such as an optical or metal wire or waveguide including a carrier wave that transmits a signal designating program instructions, a data structure, and the like. Examples of program instructions include not only machine language codes such as those generated by a compiler, but also include high-level language codes that can be executed by a computer using an interpreter or the like.

The hardware devices described above may be configured to operate as one or more software modules to perform operations of the present disclosure, and vice versa.

Although the exemplary embodiments of the present disclosure have been described in detail with reference to the accompanying drawings, it is to be understood that the present disclosure is not limited to those exemplary embodiments and various changes and modifications may be made without departing from the scope of the present disclosure. Therefore, the exemplary embodiments disclosed in the present disclosure are intended to illustrate rather than limit the scope of the present disclosure, and the scope of the technical idea of the present disclosure is not limited by these exemplary embodiments. Therefore, it should be understood that the above-described exemplary embodiments are illustrative in all aspects and not restrictive. The scope of the present disclosure should be construed according to the claims, and all technical ideas in the scope of equivalents should be construed as falling within the scope of the present disclosure.

### DESCRIPTION OF REFERENCE NUMERALS

100: device for determining preference, 110: receiving unit, 120: input unit, 130: output unit, 140: storage unit, 150: processor, 200: HMD device, 1000: system for determining preference, 302a, 302b, 302c, 302d, 302e, 302f, 302g, 302h, 302i: plurality of time units
[National R&D projects that supported this invention]
[Assignment unique number] 1711093794
[Department name] Ministry of Science and ICT
[Research Management Professional Institution] Giga Korea Foundation
[Research Project Name] Giga KOREA Project
[Research Study Name] Development and demonstration of 5G-based interactive immersive media technology
[Contribution rate] 1/1
[Organizing Agency] SK Broadband Co., Ltd.
[Study period] 20190101˜20191231

1. A method for determining preference implemented by a processor, comprising:
providing image content to a user;
receiving electroencephalogram (EEG) data and gaze data including a series of gaze position data or gaze speed data which is measured while the content is provided;
determining the user's region of interest with respect to the content based on the gaze data;
determining a saccade onset time based on the gaze data;
extracting EEG data during a time period including the saccade onset time, based on the EEG data; and
determining whether the user prefers the region of interest based on the EEG data during the time period.

2. The method of claim 1, wherein the gaze data includes the gaze position data and the gaze speed data,
wherein the determining of the saccade onset time further includes,
dividing the gaze position data into a plurality of unit periods having a predetermined time interval; and
determining a saccade period including the saccade onset time in which the user's gaze rapidly changes among the plurality of unit periods, based on gaze speed data in each of the plurality of unit periods,
wherein the extracting of the EEG data includes extracting EEG data corresponding to the saccade period.

3. The method of claim 2, wherein the determining of the saccade period includes,
classifying each of the plurality of unit periods into the saccade period or a fixation period based on the gaze speed data in each of the plurality of unit periods; and
selecting the saccade period among the plurality of classified unit periods.

4. The method of claim 3, wherein the classifying into the saccade period or the fixation period further includes,
assigning a weight to at least one period among the plurality of unit periods based on the gaze speed data; and
classifying each of the plurality of unit periods into the saccade period or the fixation period based on the weight.

5. The method of claim 4, wherein the assigning of the weight includes,
classifying the plurality of unit periods into a first group or a second group having a lower gaze speed than the first group, based on the gaze speed data;
determining a reciprocal of the number of periods belonging to the first group among the plurality of unit periods as a weight for the first group and 0 as a weight for the second group; and
assigning the weights determined for each of the first group and the second group, respectively,
wherein the classifying of each of the plurality of unit periods into the saccade period or the fixation period based on the weights includes,
determining the saccade period among the plurality of unit periods which are classified into the first group based on the weight for the first group; and
determining the fixation period based on the gaze speed data for the plurality of unit periods which are classified into the second group.

6. The method of claim 1, further comprising:
filtering the EEG data based on at least one filter among a 0.5 Hz high filter, a 60 Hz stop filter, and a 1 to 10 Hz band pass filter, which is performed after the extracting of the EEG data during the time period including the saccade onset time.

7. The method of claim 1, wherein the extracting of the EEG data during the time period including the saccade onset time includes,
extracting EEG data before and after a predetermined time based on the saccade onset time.

8. The method of claim 7, wherein the determining of whether the user prefers the region of interest includes,
determining that the region of interest is preferred when the EEG data before the saccade onset time is attenuated compared to the EEG data at the saccade onset time.

9. The method of claim 7, wherein the determining of whether the user prefers the region of interest includes,
determining that the region of interest is preferred when the EEG data after the saccade onset time is attenuated compared to the EEG data at the saccade onset time.

10. The method of claim 1, further comprising:
correcting the gaze data and the EEG data, which is performed after the receiving of the gaze data and EEG data.

11. The method of claim 1, wherein the determining of whether the user prefers the region of interest further includes,
determining whether the user prefers the region of interest by using a prediction model which is configured to predict the user's preference based on the EEG data at the saccade onset time.

12. The method of claim 1, further comprising:
differently displaying and providing the region of interest, in the content, according to whether the region of interest is preferred.

13. A device for determining preference, comprising:
an output unit configured to provide image content to a user;
a receiving unit configured to receive EEG data, and gaze data including a series of gaze position data or gaze speed data which is measured while the content is provided; and
a processor configured to communicate with the receiving unit and the output unit,
wherein the processor is configured to,
determine the user's region of interest with respect to the content based on the gaze data,
determine a saccade onset time based on the gaze data,
extract EEG data during a time period including the saccade onset time, based on the EEG data, and
determine whether the user prefers the region of interest based on the EEG data during the time period.

14. The device of claim 13, wherein the gaze data includes the gaze position data and the gaze speed data,
wherein the processor is further configured to,
divide the gaze position data into a plurality of unit periods having a predetermined time interval, and
determine a saccade period including the saccade onset time in which the user's gaze rapidly changes among the plurality of unit periods, based on gaze speed data in each of the plurality of unit periods, and extract EEG data corresponding to the saccade period.

15. The device of claim 14, wherein the processor is further configured to,
classify each of the plurality of unit periods into the saccade period or a fixation period based on the gaze speed data in each of the plurality of unit periods, and
select the saccade period among the plurality of classified unit periods.

16. The device of claim 15, wherein the processor is further configured to,
assign a weight to at least one period among the plurality of unit periods based on the gaze speed data, and
classify each of the plurality of unit periods into the saccade period or the fixation period based on the weight.

17. The device of claim 16, wherein the processor is further configured to,
classify the plurality of unit periods into a first group or a second group having a lower gaze speed than the first group, based on the gaze speed data,
determine a reciprocal of the number of periods belonging to the first group among the plurality of unit periods as a weight for the first group and 0 as a weight for the second group,
assign the weights determined for each of the first group and the second group, respectively,
determine the saccade period among the plurality of unit periods which are classified into the first group based on the weight for the first group, and
determine the fixation period based on the gaze speed data for the plurality of unit periods which are classified into the second group.

18. The device of claim 13, further comprising:
a filter unit configured to filter the EEG data based on at least one filter among a 0.5 Hz high filter, a 60 Hz stop filter, and a 1 to 10 Hz band pass filter.

19. The device of claim 13, wherein the processor is configured to,
extract EEG data before and after a predetermined time based on the saccade onset time.

20. The device of claim 19, wherein the processor is configured to,
determine that the region of interest is preferred when the EEG data before the saccade onset time is attenuated compared to the EEG data at the saccade onset time.

21-24. (canceled)
